(19) United States
(12) Patent Application Publication
(10) Pub. No.: US 2008/0063664 A1
(43) Pub. Date: Mar. 13, 2008

(54) High-yield transgenic mammalian expression system for generating virus-like particles

(75) Inventors: Pei-Wen Hsiao, Taipei (TW); Ning-Sun Yang, Taipei (TW); Chang-Jen Huang, Taipei City (TW); En-Hau Lin, Taipei City (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Academia Sinica

(21) Appl. No.: 11/515,843

(22) Filed: Sep. 5, 2006

(51) Int. Cl. A61K 39/215; C12N 7/00

(52) U.S. Cl. 424/221.1; 435/235.1; 977/803

(57) ABSTRACT

The present invention provides a method utilizing mammalian expression system for generating virus-like particles (VLPs) of mammalian-hosted viruses, particularly SARS-CoV. The method of the present invention involves expression of viral structural proteins in Vero cells and thereby obtaining recombinant VLPs in the culture medium. SARS-VLPs generated by the method of the present invention are highly immunogenic and can elicit not only humoral but also cellular immune responses in a mammal.

### FIELD OF THE INVENTION

The present invention relates to a mammalian expression system for generating virus-like particles (VLPs), and uses of VLPs generated by the mammalian expression system.

### BACKGROUND OF THE INVENTION

The spread of a newly evolved coronavirus (CoV) caused a global threat of severe acute respiratory syndrome (SARS) pandemics in 2003 (Kuiken, T. et al., 2003, Lancet 362: 263-270). Coronaviruses are taxonomically classified in the order Nidovirales, based on features of their genome organization and replication strategy. As with other coronaviruses, SARS-CoV has the morphology of enveloped particles with typical peripheral projections, termed “corona” or “spikes,” surrounding the surface of a viral core (Ksiazek, T. G. et al., 2003, N Engl J Med 348: 1953-1966; Lin, Y. et al., 2004, Antivir Ther 9: 287-289). Outside the coronavirus particle core is a layer of lipid envelope containing mainly three membrane proteins, the most abundant M (membrane) protein, the small E (envelope) protein, and the S (spike) protein. The homo-trimers of the S protein collectively form the aforementioned corona, which is involved in viral binding to host receptors, membrane fusion for viral entry, cell-to-cell spread and tissue tropism of coronaviruses. The viral core inside the envelope, termed nucleocapsid, harbors a positive-strand viral genome RNA of approximately 30 kb packaged by the N (nucleocapsid) protein.

Unlike other human coronaviruses, such as HCoV-229E and HCoV-OC43, that can cause only symptoms like the common cold, SARS-CoV causes a highly transmittable, severe and virulent disease that can often be lethal in adults and especially the elderly. Research and clinical interest on SARS-CoV has grown rapidly owing to the high infectivity and mortality. There is especially an urgent need for an effective and safe vaccine against SARS-CoV to deal with possible future reemergence of the SARS epidemics.

Most antiviral vaccines currently in use contain whole viruses, either inactivated or live-attenuated. Inactivated, or killed, viruses are treated chemically or by irradiation to disable their replication and are generally safe and easy to make. While eliciting neutralizing antibodies, they are unlikely to deliver viral antigens to cytosol for cytotoxic CD8+ T lymphocytes (CTLs) activation, which is critical to defend animals from infection. Live-attenuated vaccines are significantly more potent than killed vaccines. However, live-attenuated viruses pose the risk of reversion or recombination with circulating wild type into a virulent strain. Moreover, the manufacture of vaccines based on whole viruses also carries the risk of viral escape.

To avoid the danger of using the whole virus (such as killed or attenuated viruses) as a vaccine, recombinant viral proteins have been pursued not only as research tools but also as potential advanced subunit vaccines. However, subunit vaccines are known to suffer often from poor immunogenicity, owing to incorrect folding, poor antigen presentation, or difference in carbohydrate and lipid composition. Virus-like particles (VLPs) are self-assembled microscopic antigenic structures that resemble a virus in size and shape but lack genetic materials. VLPs can concurrently present viral proteins, carbohydrates and lipids in a similar and authentic conformation and thus have been viewed as an ideal vaccine against viruses (McGuigan, L. C. et al., 1993, Vaccine 11: 675-678). VLPs display intact viral antigens on the surface in a repeated arrangement, with which they afford the natural binding of a large viral entity to membrane receptors of antigen-presenting cells (APCs), such as dendritic cells (DCs). DC-targeted uptake of VLPs enables potent stimulation of CD4+ T cells against VLP-associated antigens. Besides stimulating humoral immunity, VLPs are permissive for cross-presentation in DCs that allows priming of CTL response with VLP-associated antigens (Moron, G. et al., 2002, J Exp Med 195: 1233-45).

VLPs for over thirty different viruses have been generated in insect and mammalian systems for vaccine purpose (Noad, R. and Roy, P., 2003, Trends Microbiol 11: 438-44). It has been shown that cellular expression of the M protein accompanied by the E protein of coronaviruses was a minimal requirement and sufficient for the assembly of VLPs (Vennema, H. et al., 1996, EMBO J 15: 2020-2028). While being dispensable in forming VLPs, the S protein can be integrated into the VLPs whenever available (Godeke, G. J. et al., 2000, J Virol 74: 1566-1571).

Researchers have used baculovirus expression systems to produce SARS VLPs (Ho, Y. et al., 2004, Biochem Biophys Res Commun 318: 833-838; Mortola, E. and Roy, P., 2004, FEBS Lett 576: 174-178). However, due to the intrinsic differences between insect cells and mammalian cells, the VLPs assembled in the insect (SF9) cells exhibited a size of 110 nm in diameter, which is much larger than the 78 nm of the authentic SARS-CoV virions (Lin, Y. et al., 2004, supra, and Ho, Y. et al., 2004, supra). Moreover, immunogenicity of the insect cell-based SARS-VLP remains uninvestigated. Other researchers also tried to use mammalian expression systems to produce SARS VLPs (Huang, Y. et al., 2004, J Virol 78: 12557-65). However, the extracellular release of VLPs is not efficient, and the yield of VLPs is not satisfying.

Therefore, there is still a need for an efficient method for the large-scale production of SARS VLPs in order to provide an effective and safe vaccine against SARS.

### BRIEF SUMMARY OF THE INVENTION

The present invention provides an efficient method for generating VLPs, wherein the generated VLPs are highly immunogenic and can serve as a useful vaccine; particularly SARS VLPs for use as a vaccine against SARS.

In some embodiments of the present invention, there is provided a method for generating virus-like particles (VLPs) of a mammalian-hosted virus, the method comprising:

constructing a plasmid comprising a nucleotide sequence encoding a combination of at least two structural proteins of the virus;

transfecting Vero cells with the plasmid; and

expressing the viral structural proteins in the transfected cells to generate VLPs of the virus.

In other embodiments of the present invention, there is provided a method for generating antibodies against SARS-CoV, comprising immunizing a mammal or bird with SARS-VLPs generated according to the present invention, and harvesting antibodies against the VLPs from the blood of the mammal or bird.

In further embodiments of the present invention, there is provided a method for detecting an infection of SARS-CoV in a subject, comprising contacting a serum sample from the subject with SARS-VLPs generated according to the present invention, and determining the presence in the sample of an antibody/antigen complex, whereby the presence of the complex indicates a positive result.

In further embodiments of the present invention, there is provided a method for detecting an infection of SARS-CoV in a subject, comprising contacting a tissue sample from the subject with antibodies against the SARS-VLPs generated according to the present invention, and determining the presence in the sample of an antibody/antigen complex, whereby the presence of the complex indicates a positive result.

In still other embodiments of the present invention, there is provided a method for preventing an infection of SARS-CoV in a subject, comprising immunizing the subject with SARS-VLPs generated according to the present invention.

In still other embodiments of the present invention, there is provided an immunogenic composition comprising SARS-VLPs generated according to the present invention.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 comprises FIGS. 1A and 1B. FIG. 1A comprises an illustration of the construction of the fluorescent SARS VLP-expressing plasmid. FIG. 1B comprises fluorescent images showing the locations of the expressed VLPs. Regarding FIG. 1A, two tet operator-regulated, CMV promoter-driven expression cassettes were constructed into the same plasmid for inducible expression of M-GFP fusion protein (i.e., the M protein fused with a green fluorescent protein (GFP)) and E protein from one cassette, and S protein from the other. FIG. 1B shows the results of the expression and assembly of fluorescent SARS VLPs in the VeroE6/S-MG-E-55 producer cell line, wherein cells were induced by adding 1 μg/ml doxycycline (Dox) to culture medium for 1 day, fixed, and then stained indirectly with antibodies specifically against M, GFP, S and E proteins as marked. The green fluorescence from GFP in the stained cells was scanned and merged for co-localization with different proteins contained in the VLP inside the producer cells.

FIG. 2 comprises FIGS. 2A-2D, and shows the results of the purification and characterization of Vero E6-secreted SARS-VLPs. Regarding FIG. 2A, secreted VLPs were purified by sucrose gradient ultra-centrifugation. Protein concentration (measured by Bradford Assay) and GFP fluorescence level in each fraction were plotted as marked. Regarding FIG. 2B, proteins contained in each fraction were analyzed by SDS-PAGE and Coomassie blue staining. Regarding FIG. 2C, identities of the protein bands marked in FIG. 2B were verified by western blot analysis using antibodies against S, M, E, or GFP proteins. FIG. 2D is an electron microscopic image of negatively stained SARS-VLPs (fractions 9 to 15 of FIG. 2B) purified by sucrose gradient from cell culture medium (the bar indicates a scale of 50 nm).

FIG. 3 comprises FIGS. 3A-3E and shows the results of immunization with SARS-VLPs induced humoral immune responses in mice. Regarding FIG. 3A, a diagram of immunization protocol, groups of four mice were subcutaneously injected with different dosage of SARS-VLPs at two time points as marked. Serum samples were examined for VLP-specific antibody responses in tested mice by ELISA after serial dilution. FIG. 3B shows graphs relating to ELISA titers of VLP-specific IgG, IgG1, and IgG2a using SARS-VLP as the capture antigen. Serum samples were collected on the 28th day after primary immunization. Dilution of test samples is marked on the X-axis. The background-subtracted absorbance (450 nm) was plotted as means±standard deviations (error bar). Presented data summarize the results of three different experiments. FIG. 3C is a graph that relates to cross-reaction of VLP-specific IgG antibodies with real SARS-CoV. Anti-sera as shown in FIG. 3B were diluted (1:250) in PBS. The SARS-specific antibody titer elicited by SARS-VLP vaccination was detected by a commercial SARS ELISA test kit (Euroimmun) according to the manufacture's protocol, except for a modification by replacing the anti-human IgG secondary antibody with anti-mouse IgG. Mean titer and standard deviation in each group of immunized mice was summarized and plotted as means±standard deviations. FIG. 3D is a graph relating to a time course of VLP-elicited antibody responses. Serum samples were collected from immunized mice at the indicated time points. Anti-sera were diluted (1:250) in PBS and titers of VLP-specific IgG were measured by ELISA analysis as in FIG. 3B. FIG. 3E relates to antigen determinants of VLP-elicited antibodies. Three doses (100, 10, 1 ng) of purified VLP were loaded as western blot antigens. Anti-sera as shown in FIG. 3B were diluted (1:1000) in PBS and subjected to western blot analysis.

FIG. 4 comprises FIGS. 4A and 4B and relates to immunization with SARS-VLPs induced cellular immune responses in mice. Primary culture of splenocytes obtained from tested mice 28 days after priming as shown in FIG. 3B were re-stimulated with SARS-VLP for 40 hours. Responsive cells that secrete Interferon-γ (FIG. 4A) and interleukin-4 (FIG. 4B) were determined by ELISPOT assays. Presented data summarize the results of three different experiments as means±standard deviations (error bar).

### DETAILED DESCRIPTION OF THE INVENTION

To generate VLPs as a SARS vaccine, technical challenges include mammalian post-translational modifications and correct folding of viral proteins, their delicate organization into a lipid envelope, and sustainable yield for practical usage. The SARS-S protein is deduced as a huge glycoprotein containing 1255 aa residues with 23 putative N-linked glycosylation sites, and at least 12 N-glycans have been identified (Krokhin, O. et al., 2003, Mol Cell Proteomics 2: 346-56). In SARS-CoV infected cells and purified virion, protein M contains one high-mannose type N-glycan (Voss, D. et al., 2006, FEBS Lett 580: 968-73). Thus, mammalian expression and cell culture-based approaches are of interest to the inventors to attain massive production of SARS-VLPs.

In one aspect, the present invention provides a method for generating virus-like particles (VLPs) of a mammalian-hosted virus, such as SARS-CoV, the method comprising:

constructing a plasmid comprising a nucleotide sequence encoding a combination of at least two structural proteins of the virus;

transfecting Vero cells with the plasmid; and

expressing the viral structural proteins in the transfected cells to generate VLPs of the virus.

The method of the present invention is suitable for generating various mammalian-hosted viruses, including but not limited to arenaviruses, coronaviruses, hepadnaviruses, herpes viruses, orthomyxoviruses, paramyxoviruses, papovaviruses, parvoviruses, and retroviruses. In a preferred embodiment of the present invention, the mammalian-hosted virus is a coronavirus. More preferably, the mammalian-hosted virus is SARS-CoV.

The term “viral structural protein” or “structural protein of a virus” and equivalent terms as used herein refers to viral genome-encoded proteins that form the structure of a virus, including membrane glycoproteins and capsid proteins. The genome of a virus also encodes non-structural regulatory proteins involved in virus replication. For example, the structural proteins of a coronavirus comprise the M (membrane), E (envelope), S (spike) and N (nucleocapsid) proteins.

In an embodiment of the method used to generate SARS-VLPs according to the invention, the structural proteins to be expressed in transfected cells can be any combinations derived with the E, M, N and S proteins of SARS-CoV, such as, for example, M+E, M+E+S, M+S, N+M+E, N+M+E+S, and N+M+S. In a preferred embodiment, the combination of the structural proteins is M+E. Most preferably, the combination of the structural proteins is M+E+S.

The plasmid used in the present invention can be any plasmid or vector suitable for expressing heterologous proteins in mammalian cells. Many commercially available mammalian expression vectors can be readily used in the present invention, for example, the pcDNA™ series by Invitrogen Corporation (Carlsbad, Calif., USA).

To construct the recombinant plasmid used in the present invention, nucleotide sequences encoding a combination of the viral structural proteins can be grouped into one or more “expression cassettes” for controlled expression. As used herein, the term “expression cassette” refers to a nucleic acid construct, generated recombinantly or synthetically, with a series of specified nucleic acid elements which permit transcription of a nucleotide sequence of interest in a host cell. The expression cassette can be incorporated into a plasmid or chromosome. Typically, the expression cassette portion of an expression vector includes, among other sequences, a nucleotide sequence to be transcribed, a promoter, and a poly-adenylation signal. In the present invention, the term “expression cassette” is used interchangeably with the term “transgene.”

For optimal expression of the viral proteins of the present invention, the expression cassette may include an inducible system that allows high-level expression upon induction. In a preferred embodiment of the present invention, a tetracycline-inducible expression system is utilized for high-level expression of the viral proteins, wherein the induction is achieved by the addition of doxycycline into the culture medium. Examples of commercially available inducible expression systems include but not limited to the T-REx™ System and GeneSwitch™ System by Invitrogen Corporation, and the BD Tet-On™ and BD Tet-Off™ Gene Expression Systems by Clontech Laboratories, Inc. (Mountain View, Calif., USA).

According to an embodiment of the present invention, the cells used in the generation of VLPs are Vero cells. The Vero cell line, i.e. the cell line of ATCC No. CCL-81™, was initiated from the kidney of a normal adult African green monkey on Mar. 27, 1962, by Y. Yasumura and Y. Kawakita at the Chiba University in Chiba, Japan. The cell line was brought to the Laboratory of Tropical Virology, National Institute of Allergy and Infectious Diseases, National Institutes of Health in the 93rd passage from Chiba University by B. Simizu on Jun. 15, 1964. In addition to its use as a vaccine cell substrate, this cell line has been used extensively for virus replication studies and plaque assays. In the present invention, the term “Vero cell” includes not only cells from the original Vero cell line, but also those derived from Vero-derived cell lines such as Vero 76 (ATCC No. CRL-1587™) and Vero E6 (ATCC No. CRL-1586™).

Transfection can be performed by any known method and can result in either transient or stable transfection. Stable transfection is preferred to establish a cell line producing VLPs of interest. Methods for obtaining stable transfection are well known and include, for example, selection for spontaneously stable transfectants, transfection with immortalizing genes, and selection for genes providing resistance to antibiotics such as neomycin, puromycin, zeocin, hygromycin B, and blasticidin S.

As demonstrated in the following examples, SARS-VLPs generated by the method of the present invention can induce high titers of SARS-CoV-specific antibodies in mice. Therefore, the present invention also provides a method for generating antibodies against SARS-CoV, comprising immunizing a mammal or bird with SARS-VLPs generated according to the present invention, and harvesting antibodies against the VLPs from the blood of the mammal or bird.

According to the following examples, in addition to eliciting humoral immune responses, SARS-VLPs generated by the method of the present invention also stimulates systemic activation of T helper (T_{H}) cells. Therefore, the present invention also provides a method for preventing an infection of SARS-CoV in a subject, comprising immunizing the subject with SARS-VLPs generated according to the present invention. Preferably, the subject is a mammal, such as a dog, a cat, a rabbit, a rat, a mouse, a pig, a sheep; a goat, and a cow, and more preferably, a human.

Immunization can be performed traditionally. Suitable regimes for initial administration and booster doses are variable, but may include an initial administration followed by subsequent booster administrations. The quantity of SARS-VLPs to be administered depends on the subject to be immunized, including, for example, the capacity of the individual's immune system to synthesize antibodies, and if needed, to produce a cell-mediated immune response. Precise amounts of VLPs required to be administered depend on the judgment of the practitioner. However, suitable dosage ranges are readily determinable by one skilled in the art without undue experimentation in view of the present disclosure. The dosage may also depend on the route of administration and will vary according to the size of the host. Non-limiting exemplary dosages include, for instance, a preferred dosage of about 0.01 mg/kg to about 10 mg/kg body weight, and a more preferred dosage of about 0.1 mg/kg to about 1 mg/kg body weight.

In another aspect, the present invention provides a method for detecting an infection of SARS-CoV in a subject, comprising contacting a serum sample from the subject with SARS-VLPs generated according to the present invention, and determining the presence in the sample of an antibody/antigen complex, whereby the presence of the complex indicates a positive result.

Preferably, the method involves an immunoassay. In a particularly preferred embodiment of the present invention, the method involves an enzyme-linked immunosorbent assay (ELISA). In ELISA assays, the VLPs are immobilized onto a selected surface, for example, a surface capable of binding proteins, such as the wells of a polystyrene microtiter plate. After washing to remove incompletely adsorbed material, a nonspecific protein, such as a solution of bovine serum albumin (BSA) that is known to be antigenically neutral with regard to the test sample may be bound to the selected surface. This allows for blocking of nonspecific adsorption sites on the immobilizing surface and thus reduces the background caused by nonspecific binding of proteins in the antisera onto the surface.

The immobilizing surface is then contacted with a sample, such as a serum sample from a subject suspected of a SARS-CoV infection, in a manner conducive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for about 2 to about 4 hours, at suitable incubation temperatures, such as of the order of about 25° C. to about 37° C. Following incubation, the sample-contacted surface is washed to remove non-immunocomplexed material. The washing procedure may include washing with a solution, such as PBS/Tween™ or a borate buffer. Following formation of specific immunocomplexes between the test sample and the bound protein, and subsequent washing, the occurrence, and even the amount, of immunocomplex formation may be determined by subjecting the immunocomplex to a second antibody having specificity for the first antibody. If the test sample is of human origin, the second antibody is an antibody having specificity for human immunoglobulins and in general IgG. To provide for the detection, the second antibody may have an associated activity such as an enzymatic activity that will generate, for example, a color development upon incubating with an appropriate chromogenic substrate. Quantification may then be achieved by measuring the degree of color generation using, for example, a spectrophotometer.

The present invention also provides another method for detecting an infection of SARS-CoV in a subject, comprising contacting a tissue sample from the subject with antibodies against the SARS-VLPs generated according to the present invention, and determining the presence in the sample of an antibody/antigen complex, whereby the presence of the complex indicates a positive result.

Preferably, the method involves an immunoassay. In a particularly preferred embodiment of the present invention, the method involves indirect immunofluorescence staining. Indirect immunofluorescence staining involves intracellular staining of specific proteins with antibodies and tracking of the signals via respective fluorescence-labeled second antibodies. For example, target cells were first fixed, permeated, and washed, and the cells were blocked with 1% gelatin/PBST for 1 hour and then reacted with the first antibody (such as anti-S, M, E and GFP) in appropriate dilution with 1% gelatin/PBST at 4° C. for overnight. Subsequent to another three washes in PBST, the cells were incubated with the fluorescence-conjugated secondary antibody, washed and scanned under a confocal microscope.

In a further aspect, the present invention provides an immunogenic composition comprising SARS-VLPs generated according to the present invention. An immunogenic composition preferably generates immunological responses, such as antibody or T-cell responses, in a subject to whom it is administered.

SARS-VLPs generated according to the present invention can be purified after being harvested from a culture medium or cell suspension and before being used in an immunogenic composition. Any method can be used that is known to separate VLPs or viruses from surrounding proteins, lipids, nucleic acids, membranes, intact cells, and the like. Especially preferred are affinity chromatography methods; for example, an immobilized monoclonal antibody specific for SARS-VLPs can be used. Additional suitable methods are gel filtration chromatography, ion exchange chromatography, and density gradient sedimentation.

The immunogenicity of SARS-VLPs generated according to the present invention may be further improved when co-administered with adjuvants. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

For example, preferred adjuvants to enhance effectiveness of an immunogenic composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59™, containing 5% Squalene™, 0.5% Tween™ 80, and 0.5% Span™ 85 (optionally containing various amounts of MTP-PE (see below), although not required) formulated into submicron particles using a microfluidizer such as Model 1 10Y microfluidizer (Microfluidics, Newton, Mass., U.S.A.), (b) SAF™, containing 10% Squalane™, 0.4% Tween™80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont., U.S.A.) containing 2% Squalene™, 0.2% Tween™ 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass., U.S.A.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc; (6) detoxified mutants of a bacterial ADP-ribosylating toxin such as a cholera toxin (CT), a pertussis toxin (PT), or an E. coli heat-labile toxin (LT), particularly LT-K63, LT-R72, CT-Si09, PT-K9/G129; and (7) other substances that act as immunostimulating agents to enhance the effectiveness of the composition.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′-dipalmitoyl-s-n-glycero-3-hydroxyphosphoryloxy)-ethylamine (MTP-PE), etc.

Pharmaceutically acceptable salts can also be used in immunogenic compositions of the present invention. For example, mineral salts such as hydrochlorides, hydrobromides, phosphates, or sulfates, as well as salts of organic acids such as acetates, propionates, malonates, or benzoates.

Immunogenic compositions of the present invention generally contain pharmaceutically acceptable excipients, such as water, saline, glycerol, and ethanol, and may contain substances such as wetting agents, emulsifying agents, or pH buffering agents.

Immunogenic compositions of the present invention may be prepared as indictable, as liquid solutions, suspensions or emulsions, and administered parenterally, by injection subcutaneous, intradermal or intramuscularly injection. Alternatively, the immunogenic compositions of the present invention may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories and oral formulations may be desirable. Oral formulations can take the form of solutions, suspensions, tablets, pills, capsules, sustained release formulations or powders.

The immunogenic composition of the present invention may further comprise antigens from other pathogens to be a multivalent immunogenic composition.

The present invention is further illustrated by the following examples, which are provided for the purpose of demonstration rather than limitation.

### EXAMPLE 1

### Expression and Assembly of SARS-VLPs

Cell Lines and Plasmids

Vero E6 cells were obtained from American Type Culture Collection (ATCC No. CRL-1586™) and routinely cultured in MEM medium supplemented with 10% fetal bovine serum. Vero E6-based tetracycline-inducible founder cells, Vero/TR, were derived by a stable transfection with the pcDNA6/TR plasmid (Invitrogen). Inducible M-GFP and E expression cassettes were constructed by PCR linking consecutively a β-globin/IgG chimeric intron (from pCI vector, Promega), M-GFP coding sequence, an internal ribosome entry site (IRES) from the encephalomyocarditis virus (ECMV), and an E coding sequence, and the construct was then inserted into the backbone of the pcDNA4/TO plasmid (Invitrogen). Inducible S expression cassette was constructed by inserting a cDNA of the S protein of TW1 strain into the pcDNA5/TO plasmid (Invitrogen). Subsequently, the entire S expression cassette was inserted into the expression plasmid for M-GFP and E to generate the pcDNA4/TO-S-MG-E vector. The sequence of the entire plasmid was verified by DNA sequencing.

Plasmid Construction

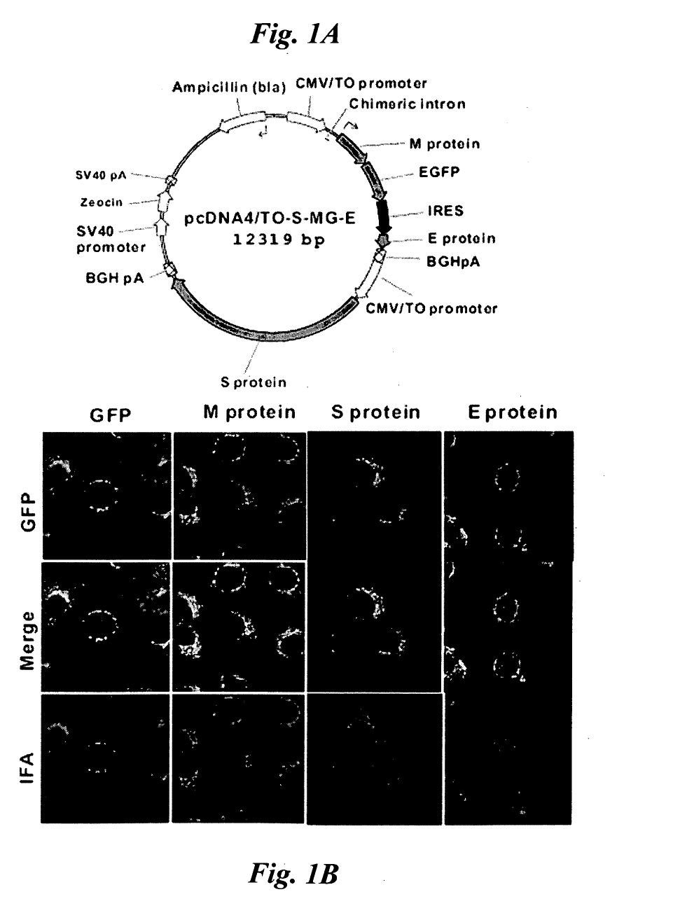

As shown in FIG. 1A, transgenes encoding the three SARS-CoV envelope proteins, S, M-GFP (i.e., the M protein fused with a green fluorescent protein (GFP) for tracking the VLPs) and E, were constructed in the same plasmid (pcDNA4/TO-S-MG-E). In one plasmid, the vector harbors two expression cassettes. The CMV/TO-MG-E cassette (SEQ ID NO: 1) transcribes an RNA transcript that holds two open-reading frames encoding the M-GFP and E proteins, which are connected by an internal ribosome entry sequence (IRES), and the CMV/TO-S cassette (SEQ ID NO: 2) expresses only the S protein. Both transcription units are regulated by a tetracycline-inducible promoter.

VLP Expression

Stable transfection of the pcDNA4/TO-S-MG-E vector into a planned Vero E6-derived founder cell line was conducted to obtain SARS-VLP expression. The founder cell has been previously stably transfected with a tetracycline repressor gene (pcDNA6/TR); therefore, the recombinant SARS-CoV genes will not express until induction. According to the fluorescence intensity of GFP, two clones were selected for prolific production of SARS-VLP, namely Vero/S-MG-E-55 and Vero/S-MG-E-68. Expression of the viral genes was induced by addition of doxycycline (1 μg/ml) to the cell culture, as verified by RT-PCR for inducible expression of RNA encoding the S, M and E (data not shown). Expression level of VLP in Vero/S-MG-E-55 is higher than Vero/S-MG-E68, therefore is primarily used.

For confocal-microscopy analysis, test cells were grown on 12 mm coverslips and treated with doxycycline (1 μg/ml) for 1 day. Cells were fixed with 4% paraformaldehyde on ice for 20 mins, permeabilized with 0.2% (v/v) Triton X™-100/PBS, and then washed with PBS three times. After blocking in 1% (v/v) fish gelatin/PBST (PBS with 0.1% Tween™-20) for 1 hr, samples were incubated with a specific antibody at 4° C. for 18 hrs, followed by 3 washes with PBST, and then probed with the respective fluorescence-conjugated secondary antibody for 1 hr at room temperature. Finally, samples were washed with PBST three times and mounted in mounting medium (Vector). The samples were scanned for GFP and antibody-stained signals, thereafter analyzed for co-localization according to the manufacture's software (Zeiss LSM 510 META).

Upon induction, GFP dots appear evident inside the producer cells within one day and accruing for longer than five days as shown in microscopic studies (FIG. 1B). The GFP dots of various sizes in the cytoplasm from the peri-nuclear region toward plasma membrane, showing indicative pattern all along the secretory pathway of mammalian cells from endoplasmic reticulum (ER) to the plasma membrane. This intracellular distribution corresponds with the CoV assembly of SARS and others, which is located at ER-Golgi-intermediate-compartment.

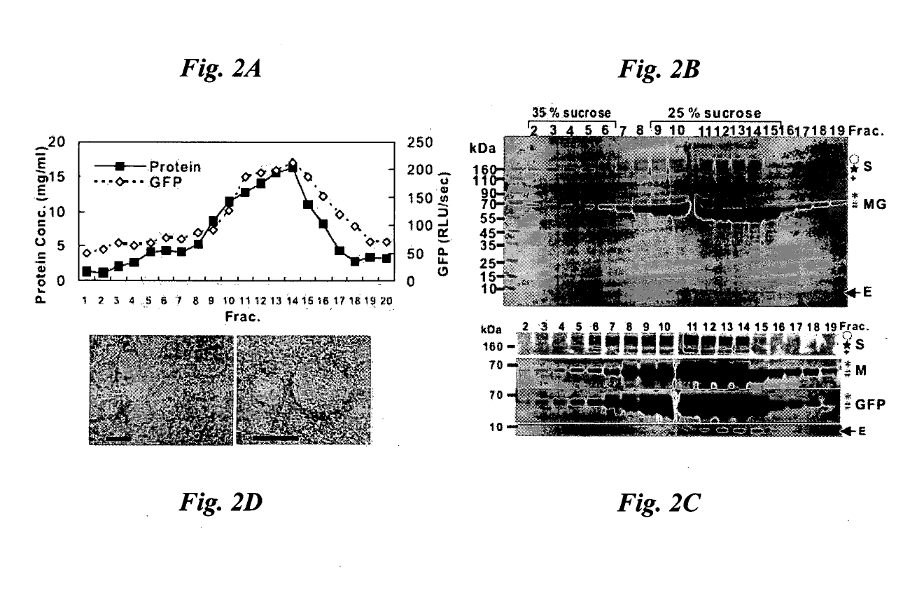

Intracellular expression of each VLP component protein (S, M, E and GFP) and their assembly were next inspected by immuno-fluorescence staining and overlaid with the fluorescence tracks of GFP, as exemplified by VLP producer cells induced for one day (FIG. 1B). Staining with antibodies against either M protein or GFP results in signals which completely overlap with GFP tracks and thus indicates that GFP fusion faithfully labels the M protein (FIG. 1B). In additional to peri-nuclear staining (Golgi complex), the S protein is stained intensely as reticular ER pattern in addition to the profiles of Golgi and secretory vesicles (FIG. 1B). However, the co-localization of S protein with M-GFP principally limits to Golgi and secretory vesicles. More S protein accumulates in ER, suggesting its longer duration for de novo synthesis and glycosylation in ER. While most secretory M-GFP dots co-localize with the staining of E protein, peri-nuclear M-GFP shows two ways, positively and negatively co-localized with E protein. These data collectively suggest that E protein as soon as it is translated initiates VLP assembly with M-GFP and S protein nearby the Golgi and resulting in punctual co-staining of M-GFP, E, and S proteins as secretory vesicles (FIG. 1B). As negative controls, the same immuno-staining with the S, M or E Ab in parental Vero E6 cells detected no signals; neither was seen for fluorescence tracks of GFP (data not shown). In agreement with previous studies on CoV budding, VLP assembly for SARS-CoV and others in mammalian cells, our data indicated the peri-nuclear assembly of SARS-M, E and S and their co-localization in a secretory vesicle profile. Assembly of the three proteins into SARS-CoV-like particles is further demonstrated by their co-sedimentation in a sucrose gradient and forming spiky spherical particles (FIG. 2D).

### EXAMPLE 2

### Purification and Characterization of SARS VLPs

Purification of VLP was initially performed by concentrating conditioned culture medium of the induced cells on a 45% sucrose cushion by ultracentrifugation at 200,000×g at 4° C. for 2 hrs. The interface was collected and further separated on a step-wise gradient between 25% and 35% sucrose at 200,000×g at 4° C., for 48 hrs. Sedimentation fractions were taken from the bottom of the tube every 0.5 ml volume. Each fraction was analyzed for protein concentration by Coomassie (Bradford) Protein Assay Kit (Pierce) and GFP fluorescence measured by VICTOR²™ fluorometer (PerkinElmer).

For western blot analysis, polyclonal antibodies against E and M proteins were separately raised in rabbits using E. coli expressed M (a.a. 53-221 of SEQ ID NO: 3) and E (a.a. 1-76 of SEQ ID NO: 4) proteins as antigens by intraspleenic injection. Anti-S polyclonal antibodies were raised in ducks using E. coli expressed S (a.a. 679-888 of SEQ ID NO: 5) as antigens, and IgY antibodies were purified from egg white (Wu, H. S. et al., 2004, J Biomed Sci 11: 117-126).

As shown in FIG. 2A, distribution of both proteins and GFP exhibited a coherent major peak concentrated in 25% sucrose layer (fractions 9 to 15). Unexpectedly, we also find a minor protein peak concentrated in 35% sucrose layer, which is absent in the Vero/S-MG-E-68 clone (fractions 2 to 6). Protein analysis by SDS-PAGE and Coomassie blue staining reveal that the two distinct peaks are obviously of different protein compositions (FIG. 2B). Each VLP constituent protein of expected size as marked in FIG. 2B is confirmed by western blot analysis using specific antibodies against S, M, E and GFP proteins (FIG. 2C). The SARS-VLP contains multiple forms of S protein, predominantly of mature form with apparent Mᵣ 180 kDa (∘), and less with 170 kDa ( [symbol] ), and 140 kDa (+) (FIG. 2B). According to previous studies on individual expression of S and M proteins in mammalian cells, the 180 kDa (∘) band represents a complex-type glycosylated form (EndoH-resistant yet PNGaseF-sensitive); the 170 kDa ( [symbol] ) band represents a high-mannose-type glycosylated form (EndoH-sensitive); and the 140 kDa (+) band represents a non-glycosylated form. The purified SARS-CoV contains two forms of M. The more abundant form with apparent Mᵣ 22 kDa is not glycosylated, and the less abundant 27 kDa form contains an EndoH-sensitive, high-mannose-type N-glycan linked to the Asn-4 residue (Voss, D. et al., 2006, supra). In agreement, the M-GFP in SARS-VLP is mainly of 65 kDa (#), and less of 70 kDa (*) (FIG. 2B). Since GFP fusion contributes Mᵣ˜27 kDa, both forms of M-GFP in SARS-VLP show an additional 16 kDa increase in apparent Mᵣ due to unknown reasons. The E protein associates with M protein in sucrose gradient sedimentation and perhaps lacks glycosylation as per its 9 kDa size.

The SARS-VLP resides in the expected major peak; whereas the unexpected minor peak comprises primarily S protein of the 170 kDa form, less for M-GFP of the 65 kDa form, but no E protein, and not observable by electron microscopy; therefore, it is not further characterized here (data not shown). The SARS-VLP of interest to the inventors (i.e., fractions 9 to 15 in FIGS. 2A-2C) contains primarily M-GFP, with less S protein and the least E protein, which is a ratio similar to CoV of SARS and others. The S protein of the secreted SARS-VLP is the predominant 180 kDa form containing complex-type N-glycans whose maturation was suggested to occur before S protein trimerizes. All forms of S protein contained in the secreted SARS-VLP were found not cleaved as estimated by their mobility in SDS-PAGE.

Morphology of the SARS-VLP was further examined by transmission electron microscopy (EM). For EM, aliquots of 10 μl of purified SARS-VLPs were loaded onto a carbon-coated grid, and let stand still for 3 mins. Grids were then stained with 2% uranyl acetate for 2 mins, and examined directly under an electron microscope. As can be seen in FIG. 2D, the negatively-stained VLP appeared as spherical particles with a spiky surface resembling SARS-CoV particle and a diameter ranging between 50 nm and 70 nm. The diameter of Vero E6 cell-secreted empty VLP is smaller than the extra-cellular whole SARS-CoV whose diameter is between 60 nm and 100 nm.

Noteworthy, the protein yield of the SARS-VLP described above is fascinatingly high, which makes the system very attractive for all relevant applications. The result demonstrated in FIG. 2 represents a routine purification of VLP from a pool of 750 ml culture medium collected on day 3 and day 5 after induction. Summation of fractions 9 to 15 (3 ml in each fraction) yields 250 mg protein of purified VLP in total (FIG. 2A). The inventors' routine yield of mammalian cell-based SARS-VLP from Vero/S-MG-E-55 cells is 449.7±69.3 (N=12) mg/L of culture medium (using 1.2×10⁸ producer cells), and is over 1,000-fold higher than the reported level of insect cell-based SARS-VLP (200 μg/L×10⁹ host cells, estimated to be 0.5 to 1 L of cell culture) (Mortola E. and Roy, P., 2004, supra). The inventors believe the unprecedentedly high expression level of SARS-VLP in this study may result from the best match of Vero E6 as host cells to express the SARS viral proteins and insertion of the transgenes into a chromatin position which is highly active in gene transcription, because the inventors also isolated many other transgenic Vero E6 clones whose intracellular expression of GFP dots were at apparently lower levels. However, it may also involve with the much stronger expression from the inducible CMV promoter used in our cell line. Production of SARS-VLPs in Vero E6 cells by stable transfection gives the best high yields to the inventors' understanding and the production process is ready to be adapted for large scale manufacture, offering an attractive approach for development of an effective and economical vaccine.

### EXAMPLE 3

### Vaccination Experiments

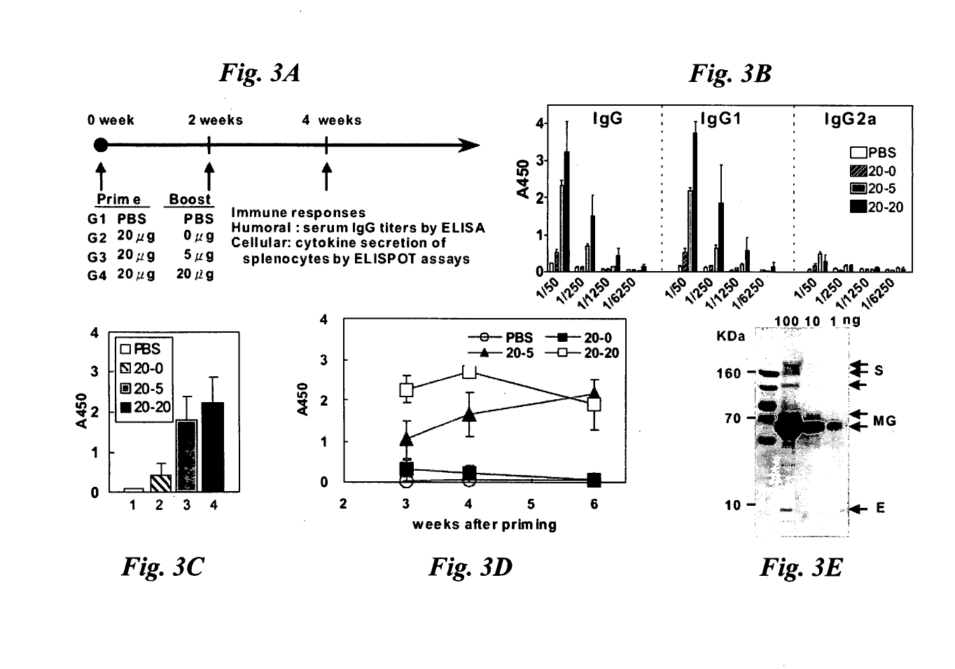

With the high-yield SARS-VLP available from mammalian expression as described above, the subsequent important question is its immunogenicity and SARS-CoV-neutralizing antibody response. To address this issue, the inventors designed a series of vaccination experiments in mice and examined the systemic immune responses (FIG. 3A). Groups of four female C57BL/6 mice, 6-8 weeks of age, were s.c. injected with 20 μg of SARS-VLP in 100 μl of PBS without additional adjuvant, and boosted with different dosages (0, 5 μg, 20 μg) after 2 weeks. Mock immunization mice were injected with 100 μl of PBS as controls.

Immunization with SARS-VLP Elicits an Antigen-specific IgG1 response in mice.

Two weeks after booster immunization, serum titers of antigen-specific IgG were measured by ELISA using native SARS-VLPs as the absorbent antigen. For ELISA, serum was collected by tail vein bleeding, allowing clotting at 4° C. overnight and cleared up by centrifugation. ELISA plates (Nunc) was coated with 1 μg native VLP at 4° C. overnight and blocked with 5% dry milk in PBS. ELISA plates were then incubated with serum samples of indicated dilution at 37° C. for 1 hr, traced with HRP-conjugated secondary antibodies, and developed color with TMB substrate (PIERCE). Washes with PBST for 5 times were applied between each step of ELISA. Finally, the ELISA was read out with absorbance of 450 nm wavelength (A₄₅₀) by a microplate reader (Power Wave XS, Bio-Teck). VLP-specific IgG titer (A₄₅₀) was calculated by subtracting the background readout of mock samples.

As shown in FIG. 3B, a single dose of 20 μg VLP positively induced antibody response up to 50-fold. The specific antibody titers were dose-dependently increased by a booster immunization for over 6250-fold (FIG. 3B). Similar ELISA for various IgG subtypes detected that the antibody response mainly restricted in IgG1 subtype which generally acts on neutralization (FIG. 3B). In contrast, IgG2a subtype of VLP-specific antibody titer was very low in these experiments (FIG. 3B). Together, the response of antibody subtype indicates an induction of T_{H}2-type effector functions against the epitopes on the SARS-VLP surface. Most prominently, the IgG antibody stimulated by the SARS-VLP effectively cross-reacted with genuine SARS-CoV virion inactivated by the gamma-radiation and heat, as demonstrated by ELISA using a commercial kit advised in the World Health Organization website (FIG. 3C). The antigen-specific antibody in mice serum retains high titers for longer than 4 weeks following the booster immunization, indicating a long persistence of antibody response caused by SARS-VLP immunization (FIG. 3D). The ELISA results in FIG. 3B-3C are particularly meaningful to SARS-CoV neutralization because they discern the antibody that binds surface of VLP and whole virus. These results endorse the resemblance in surface between the VLP and intact SARS-CoV and indicate a potential neutralizing antibody response induced by SARS-VLP vaccine in mice.

SARS-VLP-induced Serum IgG Antibodies Recognize S and M Proteins.

The antigen determinants with which VLP protein the mouse anti-bodies would react were examined by western blot assay loaded with three different amounts of SARS-VLP. As shown in FIG. 3E, the VLP-specific antibody detects the most intensely against M-GFP, followed by S protein, and minimally to E protein. The VLP-specific antibody efficiently reacted with all forms of S and M proteins. The observations specify that M and S proteins in the context of SARS-VLP are much more immunogenic than E protein, which also agrees with the antibody specificity found in SARS patients.

Immunization with SARS-VLP Induces Antigen-specific T helper (T_{H}) Responses in Mice.

The type of T_{H} response upon SARS-VLP vaccination was investigated by IFN-γ and IL-4 ELISPOT (enzyme-linked immunospot) assays for commitment to secrete T_{H}1 and T_{H}2 cytokines by splenocytes. For ELISPOT assays, PVDF-bottom plates (Millipore) were coated with 0.1 ml INF-γ and IL-4 capture antibodies (1:60; R&D systems) at 4° C. overnight. After washing with PBS twice, the plates were and then blocked with 1% BSA in PBS at room temperature for 4 hrs. Splenocytes were isolated from tested mice 14 days after booster administration, and allowing erythrocyte lysis. Splenocytes of single cell were suspended in RPMI containing 10% heat-inactive FBS, 50 μM β-mercapto-ethanol, and 3×10⁵ cells/well were grown in INF-γ or IL-4 ELISPOT plates with 1 μg VLP for 40 hrs. Washes with PBST for 5 times were applied between each step of ELISPOT. The plates were incubated with 0.1 ml biotinylated INF-γ or IL-4 detection antibodies of 1/60 dilution (R&D systems) at 4° C. overnight, incubated with streptavidin-alkaline phosphatase of 1/60 dilution (R&D systems) at room temperature for 1.5 hrs, washed, and rinsed twice with water. The color of ELISPOT was developed in darkness for 30 mins with BCIP/NBT solution (R&D systems). Development was stopped by washing with water and air-dried. The signals were counted by ImmunoSpot analyzer and analyzed by ImmunoSpot software (CTL).

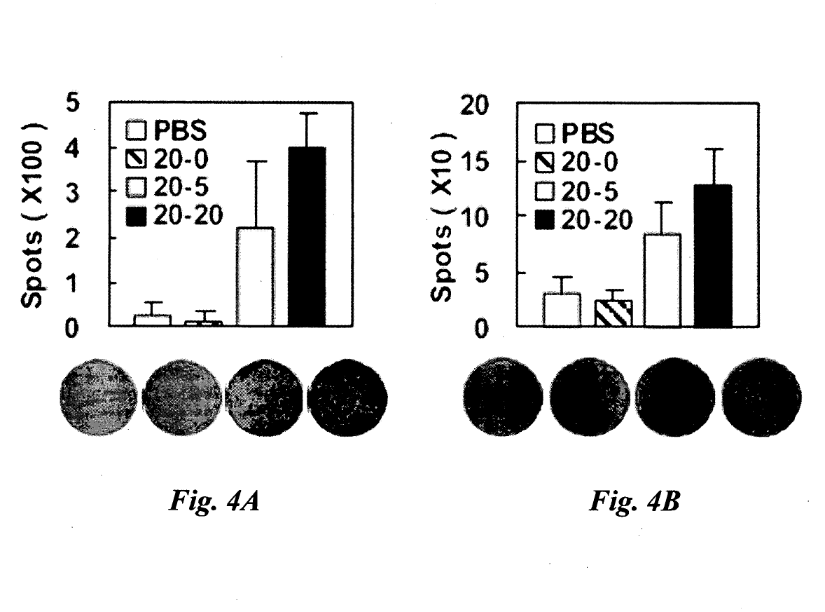

When the primary culture of splenocytes isolated from SARS-VLP-immunized mice re-exposed to SARS-VLP ex vivo, both INF-γ- and IL-4-producing populations rise along with the booster dose of SARS-VLP, indicating development of VLP-recognizing T_{H}1 cells and T_{H}2 cells in spleen provoked by SARS-VLP vaccination dose-dependently in vivo (FIGS. 4A, 4B). However, a T_{H}2-biased Ab response as indicated by induction of IgG1-dominant antibodies in serum further indicates the effector function of T_{H}1 cells in vaccinated mice was to activate CTL. Further, both T_{H}1 and CTL can secrete INF-γ when DC presents them against the VLP-antigens (FIGS. 3B, 3C). Together, these data demonstrate that SARS-VLP per se is a potent vaccine that raised humoral and cellular immune responses.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for generating virus-like particles (VLPs) of a mammalian-hosted virus, the method comprising:
constructing a plasmid comprising a nucleotide sequence encoding a combination of at least two structural proteins of the virus;
transfecting Vero cells with the plasmid; and
expressing the viral structural proteins in the transfected cells to generate VLPs of the virus.

2. The method according to claim 1, wherein the mammalian-hosted virus is a coronavirus.

3. The method according to claim 2, wherein the coronavirus is severe acute respiratory syndrome coronavirus (SARS-CoV).

4. The method according to claim 3, wherein the viral structure proteins are selected from the group consisting of E, M, N and S proteins of SARS-CoV.

5. The method according to claim 4, wherein the viral structure proteins are the E, M and S proteins of SARS-CoV.

6. The method according to claim 1, wherein the Vero cells for transfection are Vero E6 cells.

7. The method according to claim 1, wherein expression of the viral structural proteins in the transfected cells is controlled by an inducible expression system.

8. The method according to claim 7, wherein the inducible expression system is a tetracycline-inducible expression system.

9. The method according to claim 8, wherein the induction is achieved by adding doxycycline into the culture medium of the transfected cells.

10. An immunogenic composition against a mammalian-hosted virus comprising an immunoeffective amount of the VLPs generated by the method according to claim 1.

11. The immunogenic composition according to claim 10, wherein the mammalian-hosted virus is a coronavirus.

12. A vaccine composition against a mammalian-hosted virus comprising an immunoeffective amount of the immunogenic composition according to claim 10.

13. The vaccine composition according to claim 12, wherein the mammalian-hosted virus is a coronavirus.

14. A method for generating antibodies against SARS-CoV, comprising immunizing a mammal or bird with SARS-VLPs generated by the method according to claim 3, and harvesting antibodies against the VLPs from the blood of the mammal or bird.

15. A method for detecting an infection of SARS-CoV in a subject, comprising contacting a serum sample from the subject with SARS-VLPs generated by the method according to claim 3, and determining the presence in the sample of an antibody/antigen complex, whereby the presence of the complex indicates a positive result.

16. The method according to claim 15, wherein the method involves an enzyme-linked immunosorbent assay (ELISA).

17. A method for detecting an infection of SARS-CoV in a subject, comprising contacting a tissue sample from the subject with antibodies against the SARS-VLPs generated by the method according to claim 3, and determining the presence in the sample of an antibody/antigen complex, whereby the presence of the complex indicates a positive result.

18. The method according to claim 17, wherein the method involves an indirect immuno-fluorescence staining assay.

19. A method for preventing an infection of SARS-CoV in a subject, comprising immunizing the subject with SARS-VLPs generated by the method according to claim 3.

20. An immunogenic composition comprising an immunoeffective amount of SARS-VLPs generated by the method according to claim 3.

21. A vaccine against SARS, comprising the immunogenic composition of claim 20.


SEQUENCE LISTING (5 sequences)

SEQ ID NO: 1 - DNA, 3484 bp, Artificial
  expression cassette
  ref <301>: AUTHORS: Yao, F., Svensjo, T., Winkler, T., Lu, M., Eriksson, C., and Eriksson, E.
  ref <302>: TITLE: Tetracycline Repressor, tetR, Rather than the tetR-Mammalian Cell Transcription Factor Fusion Derivatives, Regulates Inducible Gene Expression in Mammalian Cells
  ref <303>: JOURNAL: Hum. Gene Ther.
  ref <304>: VOLUME: 9
  ref <305>: ISSUE: 13
  ref <306>: PAGES: 1939-1950
  ref <307>: DATE: 1998-09-01
  ref <313>: RELEVANT RESIDUES: (1)..(770)
  ref <308>: DATABASE ACCESSION NUMBER: Entrez Nucleotide / AY278741
  ref <309>: DATABASE ENTRY DATE: 2005-10-04
  ref <313>: RELEVANT RESIDUES: (1004)..(1666)
  ref <308>: DATABASE ACCESSION NUMBER: Entrez Nucleotide / AY278741
  ref <309>: DATABASE ENTRY DATE: 2005-10-04
  ref <313>: RELEVANT RESIDUES: (2992)..(3222)
  ref <308>: DATABASE ACCESSION NUMBER: Entrez Nucleotide / U57609
  ref <309>: DATABASE ENTRY DATE: 2003-08-29
  ref <313>: RELEVANT RESIDUES: (1673)..(2389)
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatcccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacaggagctcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccgccctcgtgaccaccctgacctacggcgtgcagtgcttgagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattccgcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaaacgtctaggccccccgaaccacggggacgtggttttcctttgaaaaacacgatgataatatggccacaaccggatctatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 2 - DNA, 4837 bp, Artificial
  expression cassette
  ref <301>: AUTHORS: Yao, F., Svensjo, T., Winkler, T., Lu, M., Eriksson, C., and Eriksson, E.
  ref <302>: TITLE: Tetracycline Repressor, tetR, Rather than the tetR-Mammalian Cell Transcription Factor Fusion Derivatives, Regulates Inducible Gene Expression in Mammalian Cells
  ref <303>: JOURNAL: Hum. Gene Ther.
  ref <304>: VOLUME: 9
  ref <305>: ISSUE: 13
  ref <306>: PAGES: 1939-1950
  ref <307>: DATE: 1998-09-01
  ref <313>: RELEVANT RESIDUES: (1)..(770)
  ref <308>: DATABASE ACCESSION NUMBER: Entrez Nucleotide / AY291451
  ref <309>: DATABASE ENTRY DATE: 2004-02-25
  ref <313>: RELEVANT RESIDUES: (771)..(4538)
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaaagcttgcaatcactagtgaattcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 3 - protein, 221 aa, SARS coronavirus Urbani
  ref <308>: DATABASE ACCESSION NUMBER: Entrez Protein / AAP13444
  ref <309>: DATABASE ENTRY DATE: 2005-10-04
  ref <313>: RELEVANT RESIDUES: (1)..(221)
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHPLGRCDIKDLPKEITVATSRTLSYYKLGASQRVGTDSGFAAYNRYRIGNYKLNTDHAGSNDNIALLVQ

SEQ ID NO: 4 - protein, 76 aa, SARS coronavirus Urbani
  ref <308>: DATABASE ACCESSION NUMBER: Entrez Protein / AAP13443
  ref <309>: DATABASE ENTRY DATE: 2005-10-04
  ref <313>: RELEVANT RESIDUES: (1)..(76)
MYSFVSEETGTLIVNSVLLFLAFVVFLLVTLAILTALRLCAYCCNIVNVSLVKPTVYVYSRVKNLNSSEGVPDLLV

SEQ ID NO: 5 - protein, 1255 aa, SARS coronavirus TW1
  ref <308>: DATABASE ACCESSION NUMBER: Entrez Protein / AAP37017
  ref <309>: DATABASE ENTRY DATE: 2004-02-25
  ref <313>: RELEVANT RESIDUES: (1)..(1255)
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT